(19) United States
(12) Patent Application Publication
(10) Pub. No.: US 2011/0306663 A1
(43) Pub. Date: Dec. 15, 2011

(54) TRIHEPTANOIN DIET FOR ADULT POLYGLUCOSAN BODY DISEASE (APBD) TREATMENT

(75) Inventors: Raphael Schiffmann, Rockwall, TX (US); Fanny Mochel, Dallas, TX (US)

(73) Assignee: Baylor Research Institute, Dallas, TX (US)

(21) Appl. No.: 13/159,329

(22) Filed: Jun. 13, 2011

Related U.S. Application Data
(60) Provisional application No. 61/354,472, filed on Jun. 14, 2010.

(51) Int. Cl. A61K 31/225; A61P 3/00

(52) U.S. Cl. 514/547

(57) ABSTRACT

Compositions and methods for the treatment and management of adult polyglucosan body disease (APBD) are disclosed herein. The APBD patients studied in the present invention experienced stabilization of disease progression and limited functional improvement with dietary triheptanoin (C7TG). The amount of C7TG administered to the patient daily for 6-8 months was 1-2 g/kg/24 hrs. The present invention demonstrates, for the first time, the arrest of clinical deterioration with limited functional recovery in APBD with triheptanoin diet therapy.

### CROSS-REFERENCE TO RELATED APPLICATIONS

This patent application is a non-provisional application of U.S. provisional patent application 61/354,472 filed on Jun. 14, 2010 and entitled “Triheptanoin Diet for Adult Poylglucosan Body Disease (APBD) Treatment” which is hereby incorporated by reference in its entirety.

### TECHNICAL FIELD OF THE INVENTION

The present invention relates in general to the field of treatment agents for metabolic disorders, and more particularly to the use of diet comprising triheptanoin for the treatment of adult polyglucosan body disease (APBD).

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with the use of therapeutic agents for the detection and treatment of disorders associated with glycogen brancher enzymes (GBE) including adult polyglucosan body disease (APBD).

U.S. Patent Publication No. 20020102737 (Millington et al. 2002) provides methods of screening subjects for lysosomal storage diseases, preferably glycogen storage diseases, using a tetrasaccharide as a biomarker. In a more preferred embodiment, subjects are screened for Pompe disease (i.e., glycogen storage disease type II). Also provided are neonatal screening assays. The present invention further provides methods of monitoring the clinical condition and efficacy of therapeutic treatment in affected subjects. Further provided are methods of measuring a tetrasaccharide biomarker by tandem mass spectrometry, preferably, as part of a neonatal screening assay for Pompe disease.

U.S. Patent Publication No. 20080085920 (Donello and Schweighoffer, 2008) describes methods and compositions for the treatment of conditions including stress-associated, chronic pain, and neurodegenerative conditions in a mammal using a composition comprising NB-DNJ or a compound structurally similar thereto. The neurodegenerative condition is selected from the group consisting of Motor Neuron Disease (ALS), Parkinsonian Syndromes, multiple sclerosis, diffuse cerebral cortical atrophy, Lewy-body dementia, Pick disease, mesolimbocortical dementia, thalamic degeneration, bulbar palsy, Huntington chorea, cortical-striatal-spinal degeneration, cortical-basal ganglionic degeneration, cerebrocerebellar degeneration, familial dementia with spastic paraparesis, polyglucosan body disease, glaucoma, Shy-Drager syndrome, olivopontocerebellar atrophy, macular degeneration, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, AIDS related dementia, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, diabetic retinopathy, Alzheimer's disease and ophthalmoplegia.

### SUMMARY OF THE INVENTION

The present invention describes the use of diet comprising triheptanoin for alleviation of symptoms, improvement of motor skills and functions and for the therapy of APBD.

The present invention is directed towards a method of alleviating symptoms, improving one or more motor skills, improving a gait, treating adult polyglucosan body disorder (APBD) or combinations thereof in a patient, comprising the steps of: identifying the patient in need of alleviation of symptoms, improvement of one or more motor skills, improvement of the gait, treatment against the APBD or combinations thereof and administering to the patient daily a dose of triheptanoin (C7TG), wherein the C7TG can optionally be mixed in with one or more food products for oral consumption by the patient. The improvement in one or more motor skills and gait are selected from the group consisting of increase in unaided walking time, time in cadence, support time, stride length, step length and walking speed.

In one aspect of the method the patient is on a regular diet, wherein the regular diet comprises one or more sources of proteins, carbohydrates, and fats. In another aspect the C7TG comprises 30-35% of a daily caloric intake of the patient. In another aspect the C7TG comprises 30%, 31%, 32%, 33%, 34%, and 35% of the daily caloric intake of the patient. In yet another aspect the amount of C7TG administered to the patient is 1-2 g/kg/24 hrs, more specifically the amount of C7TG administered to the patient is 1, 1.1, 1.2, 1.3, 1.4, 1.5, 1.6, 1.7, 1.8, 1.9, and 2.0 g/kg/24 hrs. As per the method described hereinabove he dose of C7TG is administered daily for 6-8 months.

The method of the instant invention further comprising the steps of: monitoring the progression of the therapy by measuring the levels of one or more metabolite markers of APBD in a body fluid of the patient, comparing the levels of the one or more metabolites with the levels obtained with a baseline level and a control level, wherein the baseline level is the level of the metabolites in the body fluid of the patient prior to the commencement of the treatment and the control level is the level of the metabolites in the body fluid of a healthy subject not suffering from APBD, and continuing or terminating the therapy or altering a dose, a frequency or both of the C7TG based on the results of the comparison of the metabolite levels. In one aspect the body fluid is selected from the group consisting of blood, plasma, and urine. In another aspect the C7TG is used to treat one or more disorders selected from glycogen branching enzyme deficiency disorders, Andersen disease, Forbes disease, and Danon disease.

In one embodiment the instant invention also discloses a composition for alleviating symptoms, improving one or more motor skills, improving a gait, treating adult polyglucosan body disorder (APBD) or combinations thereof in a patient comprising: triheptanoin (C7TG), wherein the C7TG is used as is or is mixed in with one or more food products for oral administration for the alleviation of symptoms, improvement of one or more motor skills, improvement of the gait, treatment against the APBD or combinations thereof in the patient; and, an optional organoleptic carrier and one or more optional additives selected from the group consisting of flavoring agents, vitamins, mineral supplements, protein supplements, coloring agents, and preservatives In one aspect the improvement in one or more motor skills and gait are selected from the group consisting of increase in unaided walking time, time in cadence, support time, stride length, step length, and walking speed.

In another aspect the composition is administered while maintaining a regular diet in the patient. In another aspect the C7TG comprises 30-35% of a daily caloric intake of the patient, more specifically the C7TG comprises 30%, 31%, 32%, 33%, 34%, and 35% of the daily caloric intake of the patient. In yet another aspect the amount of C7TG administered to the patient is 1-2 g/kg/24 hrs. In one aspect the amount of C7TG administered to the patient is 1, 1.1, 1.2, 1.3, 1.4, 1.5, 1.6, 1.7, 1.8, 1.9, and 2.0 g/kg/24 hrs, administered daily for 6-8 months. In yet another aspect the composition is used to treat one or more disorders selected from glycogen branching enzyme deficiency disorders, Andersen disease, Forbes disease, and Danon disease.

In another embodiment the present invention provides a method of alleviating symptoms, improving one or more motor skills, improving a gait, treating adult polyglucosan body disorder (APBD) or combinations thereof in a patient comprising the steps of: identifying the patient in need of alleviation of symptoms, improvement of one or more motor skills, improvement of the gait, treatment against the APBD or combinations thereof and administering to the adult patient a physiologically effective amount of a formulation orally, wherein the formulation comprises one or more odd-chain triglycerides having the general formula:

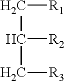

wherein, the R₁, R₂, and R₃ are esterified to the glycerol backbone are each independently fatty acids comprising odd numbered carbon chains having 5 to 15 carbon atoms, an optional organoleptic carrier, and one or more optional additives selected from the group consisting of flavoring agents, vitamins, mineral supplements, protein supplements, coloring agents, and preservatives.

In one aspect the R₁, R₂, and R₃ carbon chains are five carbons in length selected from pentanoin, triheptanoin, pentanoylcarnitine, n-pentadecanoic acid, five carbon fatty acid precursors, and derivatives thereof. In another aspect at least one of the R₁, R₂, and R₃ carbon chains are seven carbons in length. In a specific aspect the odd-chain triglyceride is triheptanoin. In yet another aspect the formulation is used to treat one or more disorders selected from glycogen branching enzyme deficiency disorders, Andersen disease, Forbes disease, and Danon disease.

Yet another embodiment of the present invention discloses a dietary composition for providing a high fat, low carbohydrate diet to a human subject comprising: one or more medium chain triglycerides (MCTs) having the general formula:

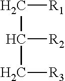

wherein, the R₁, R₂, and R₃ are esterified to the glycerol backbone are each independently fatty acids comprising odd numbered carbon chains having 5 to 15 carbon atoms; an optional organoleptic carrier; and one or more optional additives selected from the group consisting of flavoring agents, vitamins, mineral supplements, protein supplements, coloring agents, and preservatives.

In one aspect the R₁, R₂, and R₃ carbon chains are five carbons in length selected from pentanoin, triheptanoin, pentanoylcarnitine, n-pentadecanoic acid, five carbon fatty acid precursors, and derivatives thereof. In another aspect at least one of R₁, R₂, and R₃ carbon chains are seven carbons in length. In related aspects the odd-chain triglyceride is triheptanoin and the human subject is a healthy human subject or a human subject suffering from one or more glycogen brancher enzyme deficiency, adult polyglucosan body disorder (APBD), Andersen disease, Forbes disease, and Danon disease. In yet another aspect the composition is adapted for administration to a human subject suspected of having adult polyglucosan body disorder (APBD).

One embodiment discloses a dietary formulation suitable for human consumption comprising medium chain triglycerides, odd numbered carbon chain fatty acids selected from the group consisting of, five seven, and fifteen carbon fatty acids, and triglycerides thereof or both. In specific aspects the fatty acid is pentanoic acid, heptanoic acid and the odd-chain triglyceride is triheptanoin. In one aspect the composition is used to treat or alleviate the symptoms associated with one or more glycogen brancher enzyme deficiency, adult polyglucosan body disorder (APBD), Andersen disease, Forbes disease, and Danon disease. In a specific aspect the formulation is adapted for oral administration to a patient with APBD. In another aspect the formulation is adapted for enteral or parenteral administration.

Another embodiment of the present invention describes a method of treating or alleviating symptoms in an adult patient suffering from adult polyglucosan body disorder (APBD) comprising the steps of: identifying the adult patient in need of treatment or alleviation symptoms against APBD and administering a formulation of an odd-chain fatty acid comprising at least one of a C5, C7, C9, C11, C13, C15 or triglyceride thereof, to the patient in a quantity sufficient to treat or alleviate the symptoms of the APBD. In one aspect the formulation comprises one or more optional additives selected from the group consisting of flavoring agents, vitamins, mineral supplements, protein supplements, coloring agents, and preservatives. In another aspect the formulation is adapted for parenteral, enteral, intravenous or intramuscular administration.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic representation showing transport of C5-ketone bodies across the blood-brain barrier;

FIG. 2 is a plot showing the results of the 6-minutes walk tests on the five patients undergoing the Triheptanoin diet therapy according to an embodiment of the instant invention; and

FIG. 3 is a plot showing physical Functioning SF-36 scores of the five ABPD patients on the open-label triheptanoin study.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention presents results obtained in an open-label study with triheptanoin oil in 5 patients with APBD and GBE1 deficiency showed that within 6 months of treatment, patients had a significant improvement in the distance walked during 6 minutes (6 minutes walk test). Gait analysis showed stability or slight improvement over this period of time. No significant adverse events occurred. SF-36 Health Survey Questionnaire scores tended to improve in parallel with motor score.

Adult polyglucosan disease (APBD) is a progressive neurogenetic disorder characterized by onset in the 4th or 5th decade of life of neurogenic bladder and progressive difficulty walking with sensory abnormalities in the lower extremities. The motor and sensory abnormalities are caused by a myelopathy combined often with a peripheral neuropathy. After about a decade of disease progression most patients lose the ability to walk independently and in the years that follow the weakness progressively involved the trunk and the upper extremities. The disease often leads to premature death. Many of the patients with APBD suffer from an adult form of glycogen storage disease type IV (MIM 232500) cause by brancher enzyme 1 (GBE1) deficiency. The vast majority of patients with GBE 1 deficiency are of Ashkenazi Jewish (AJ) ancestry.

Overall, the frequency of all glycogen storage diseases is 1:10,000 with GBE1 deficiency constituting about 3% of all glycogen storage diseases. ABPD with GBE1 deficiency is a very rare disorder with less than 50 patients described in the English medical literature. APBD has no known effective treatment that reverses or even slows the progression of the disease. The mechanism by which GBE deficiency causes a neurological disorder is not known. One hypothesis states that the polyglucosan inclusions mechanically disrupt normal cellular function such as intra-cellular transport. The present study advances the hypothesis that decreased glycogen degradation leads to energy deficit in glia and neurons. Therefore, anaplerotic therapy, i.e. molecules providing intermediates to the citric acid cycle, may augment cellular energy production thus preventing or reversing cellular damage.

As used herein, the terms “subject” or “patient” are intended to include living organisms that may have one or more one or more glycogen brancher enzymes (GBE) deficiencies selected from Andersen disease, Forbes disease, and Danon disease, and adult polyglucosan body disorder (APBD). Examples of subjects include humans, monkeys, horses, cows, sheep, goats, dogs, cats, mice, rats, and transgenic species thereof. Other examples of subjects include experimental animals such as mice, rats, dogs, cats, goats, sheep, pigs, and cows. A subject can be a human suffering from, or suspected of having, against GBE deficiency or APBD.

As used herein, the phrases “therapeutically effective dosage” or “therapeutically effective amount” is an amount of a compound or mixtures of compounds, such as the odd-chain fatty acids and precursors or derivatives thereof, that reduce the amount of one or more symptoms of the condition in the infected subject by at least about 20%, at least about 40%, even more at least about 60%, 80% or even 100% relative to untreated subjects with a neurological or a neurodegenerative disorder. Active compounds are administered at a therapeutically effective dosage sufficient to treat a condition associated with a condition in a subject. For example, the efficacy of a compound can be evaluated in patients or animal model systems that may be predictive of efficacy in treating the disease in humans or animals.

As used herein the term, “odd-chain fatty acids” is used to describe fats and oils in foods are made up of basic units called fatty acids. In the body, these typically travel in three's as fatty acid chains attached to glycerol, forming a triglyceride. An odd-chain fatty acid that is attached to glycerol is described herein as an odd-chain triglyceride. Both the odd-chain fatty acid and the odd-chain triglyceride are part of the present invention and are often used interchangeably. For example, when referring to an odd-chain fatty acid it is possible to substitute with, or provide as, the odd-chain triglyceride and vice verse.

Based on their chemical structure, fatty acids are classified into 3 major categories: monounsaturated, polyunsaturated, or saturated fats. The oils and fats that people and animals eat are nearly always mixtures of these 3 types of fatty acids, with one type predominating. Two specific types of polyunsaturated fatty acids, linoleic and alpha-linoleic, are called essential fatty acids. They must be present in the diet in adequate amounts because they are considered necessary for proper nutrition and health. Linoleic acid (LA) is an omega-6 fatty acid and is found in many oils, e.g., corn, safflower, soybean and sunflower, whole grains and walnuts. Alpha-linoleic acid (ALA) is a plant precursor of docosahexanoic acid (DHA). Sources of ALA include seaweeds and green leaves of plants (in very small amounts), soybeans, walnuts, butternuts, some seeds (flax, chia, hemp, canola) and the oils extracted from these foods.

As used herein, the term “nutritionally effective amount” is used to mean the amount of odd-chain fatty acids and/or odd-chain triglycerides that will provide a beneficial nutritional effect or response in a mammal. For example, as with a nutritional response to vitamin- and mineral-containing dietary supplements varies from mammal to mammal, it should be understood that nutritionally effective amounts of the odd-chain fatty acids will vary. Thus, while one mammal may require a particular profile of vitamins and minerals present in defined amounts, another mammal may require the same particular profile of vitamins and minerals present in different defined amounts.

When provided as a dietary supplement or additive, the odd-chain fatty acids and/or odd-chain triglycerides of the invention has been prepared and administered to mammals in powdered, reconstitutable powder, liquid-solid suspension, liquid, capsule, tablet, caplet, lotion and cream dosage forms. The skilled artisan in the science of formulations can use the odd-chain fatty acids disclosed herein as a dietary supplement that may be formulated appropriately for, e.g., irrigation, ophthalmic, otic, rectal, sublingual, transdermal, buccal, vaginal, or dermal administration. Thus, other dosage forms such as chewable candy bar, concentrate, drops, elixir, emulsion, film, gel, granule, chewing gum, jelly, oil, paste, pastille, pellet, shampoo, rinse, soap, sponge, suppository, swab, syrup, chewable gelatin form, chewable tablet and the like, can be used.

Due to varying diets among people, the dietary odd-chain fatty acids of the invention may be administered in a wide range of dosages and formulated in a wide range of dosage unit strengths. It should be noted that the dosage of the dietary supplement can also vary according to a particular ailment or disorder that a mammal is suffering from when taking the supplement. For example, a person suffering from chronic fatigue syndrome or fibromyalgia will generally require a dose different than an athlete who is wanting to attain a nutritional benefit or obtain an increase in mental focus. An appropriate dose of the dietary supplement can be readily determined by monitoring patient response, i.e., general health, to particular doses of the supplement. The appropriate doses of the supplement and each of the agents can be readily determined in a like fashion by monitoring patient response, i.e., general health to particular doses of each.

The odd-chain fatty acids may be administered simultaneously or sequentially in one or a combination of dosage forms. While it is possible and even likely that the present dietary supplement will provide an immediate overall health benefit, such benefit may take days, weeks or months to materialize. Nonetheless, the present dietary odd-chain fatty acid supplement will provide a beneficial nutritional response in a mammal consuming it.

The odd-chain fatty acids of the present invention may be administered, e.g., orally or by subcutaneous, intravenous, intraperitoneal, etc., administration (e.g. by injection). Depending on the route of administration, the active compound may be neutralized, made miscible, at least partially or fully water-soluble or even coated in a material to protect the odd-chain fatty acids from the action of bases, acids, enzymes or other natural conditions that may interfere with their effectiveness, uptake or metabolic use.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, emulsifiers, liposomes, or a diluent. Pharmaceutically acceptable diluents include saline and aqueous buffer solutions. The therapeutic odd-chain fatty acids may be dispersed in glycerol, liquid polyethylene glycols, and mixtures thereof and in oils. Under ordinary conditions of storage and use, these preparations may contain a preservative to prevent the growth of microorganisms.

Pharmaceutical compositions that include the odd-chain fatty acids of the present invention suitable for injectable use may include sterile aqueous solutions, dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

The odd-chain fatty acids may be provided with a carrier in a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent, which delays absorption, for example, aluminum monostearate or gelatin.

The odd-chain fatty acids may be provided in one or more controlled sizes and characteristics with one or more water-soluble polymers depending on the size and structural requirements of the patient, e.g., the particles may be small enough to traverse blood vessels when provided intravenously. Either synthetic or naturally occurring polymers may be used, and while not limited to this group, some types of polymers that might be used are polysaccharides (e.g. dextran, ficoll), proteins (e.g. poly-lysine), poly(ethylene glycol), or poly(methacrylates). Different polymers, because of their different size and shape, will produce different diffusion characteristics for the odd-chain fatty acids in the target tissue or organ.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier, which contains a basic dispersion medium and the required other ingredients from those enumerated above. In the case of sterile powders for the preparation of sterile injectable solutions, methods of preparation include: vacuum drying, spray freezing, freeze-drying and the like, which yield a powder of the active ingredient (i.e., the therapeutic compound) plus any additional desired ingredient from a previously sterile-filtered solution thereof.

The odd-chain fatty acids can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. The odd-chain fatty acids may be incorporated with one or more excipients for use in, e.g., ingestible tablets, buccal tablets, troches, capsules, elixirs, suspensions, syrups, wafers, and the like. The amount of odd-chain fatty acids in the compositions and preparations may, of course, be varied depending on, e.g., the age, weight, gender, condition, disease and course of treatment of the individual patient. Pediatric doses are likely to differ from adult doses as will be known to the skilled artisan. The amount of the therapeutic compound in such therapeutically useful compositions is such that a suitable dosage will be obtained.

A dosage unit for use with the odd-chain fatty acids disclosed herein may be a single compound or mixtures thereof with other compounds, e.g., amino acids, nucleic acids, vitamins, minerals, pro-vitamins and the like. The compounds may be mixed together, form ionic or even covalent bonds. For pharmaceutical purposes the odd-chain fatty acids (e.g., C5, C7, C9, C11, C13 and/or C15) of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the odd-chain fatty acids of the present invention to a patient in need of therapy that includes a number of conditions, e.g., polysaccharide storage diseases, fatigue, low energy, wasting and the like. The odd-chain fatty acids may also be administered as any one of known salt forms.

The total daily amount of odd-chain fatty acids will vary depending on the condition and needs of a patient. For example, the odd-chain fatty acids may be provided as a supplemental source of immediate, short-term, mid-term or long-term energy and may be provided in formulations that are immediately available, slow release or extended release. The dosage amount may be measured in grams per day, as a percentage of kCalories consumed in a day, as a percentage of the total daily caloric intake, as part of a fixed, a modified or a diet that changes over time. For example, a patient may need immediate intervention that “spikes” the amount of odd-chain fatty acids to approach or reach ketosis. These “ketogenic” odd-chain fatty acids will then be varied to not have other side effects, e.g., start with 40% of total caloric intake per day and then reduced over time as the patient's condition, symptoms, clinical course and/or metabolic conditions improves. The range of percentage caloric intake may vary from between about 0.01, 0.1, 1, 2, 5, 10, 15, 20, 22, 25, 30, 35, 40 or even higher percent, which may include one or more of the odd-chain fatty acids (e.g., C5, C7, C9, C11, C13 and/or C15 (available from, e.g., Sassol, Germany). One way to measure the effect and/or dosing of the odd-chain fatty acids is to measure the amount that is detectable in body solids or fluids, e.g., biopsies and blood, respectively. A wide variety of odd-chain fatty acids metabolites may be detected from multiple sources, e.g., urine, tears, feces, blood, sweat, breath and the like.

For example, when using C7 as the source of odd-chain fatty acids these can be provided in the form of a triglyceride, e.g., tri-heptanoin. The triglyceride triheptanoin is provided in a concentration sufficient to provide a beneficial effect is most useful in this aspect of the present invention. The seven-carbon fatty acid may be provided, e.g.:

| | | |
|---|---|---|
| Infants | 1-4 g/kg | 35% kcalories |
| Children | 3-4 g/kg | 33-37% kcalories |
| Adolescent | 1-2 g/kg | 35% kcalories |
| Adults | 0.1-2 g/kg | 35% kcalories |

Goals have been set using 4 g/kg (within ideal body weight (IBW) range) for infants, children, and some adolescents. Goals have been set using 2 g/kg (within IBW range) for adolescents. Goals have been set using 2 g/kg (within IBW range) for adults; but toleration is 1-1.2 g per kg (which is 35% kcal of estimated needs).

The odd-chain fatty acids are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the odd-chain fatty acids may be formulated to provide, e.g., maximum and/or consistent dosing for the particular form for oral, rectal, topical, intravenous injection or parenteral administration. While the odd-chain fatty acids may be administered alone or pure, they may also be provided as stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions of each incorporated herein by reference.

Odd-chain fatty acids may be administered in the form of an emulsion and/or liposome, e.g., small unilamellar vesicles, large unilamallar vesicles and multilamellar vesicles, whether charged or uncharged. Liposomes may include one or more: phospholipids (e.g., cholesterol), stearylamine and/or phosphatidylcholines, mixtures thereof, and the like. Examples of emulsifiers for use with the present invention include: Imwitor 370, Imwitor 375, Imwitor 377, Imwitor 380 and Imwitor 829.

The odd-chain fatty acid vesicles may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylaspartamidephenol, or polyethyleneoxide-polylysine substituted with palmitoyl residues, mixtures thereof, and the like. Furthermore, the vesicles may be coupled one or more biodegradable polymers to achieve controlled release of the odd-chain fatty acids. Biodegradable polymers for use with the present invention include, e.g., polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the odd-chain fatty acid in its native state. For oral administration in a liquid dosage form, the oral drug components may be combined with any oral, non-toxic, pharmaceutically acceptable inert carrier such as an emulsifier, a diluent or solvent (e.g., ethanol), glycerol, water, and the like. Examples of suitable liquid dosage forms include oily solutions or suspensions in water, pharmaceutically acceptable fats and oils, alcohols or other organic solvents, including esters, emulsions, syrups or elixirs, suspensions, solutions and/or suspensions reconstituted from non-effervescent granules and even effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water-soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in multiple editions of Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the odd-chain fatty acids may also be delivered as an intranasal form via use of a suitable intranasal vehicle. For dermal and transdermal delivery, the odd-chain fatty acids may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution.

To the extent that the odd-chain fatty acids may be made into a dry powder or form, they may be included in a tablet. Tablets will generally include, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

Capsules: Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules: The odd-chain fatty acids may be dissolved in an oil, e.g., a digestible oil such as soybean oil, cottonseed oil or olive oil. Non-digestible oils may also be used to have better control over the total caloric intake provided by the oil. The active ingredient is prepared and injected by using a positive displacement pump into gelatin to form soft gelatin capsules containing, e.g., 100-500 milligrams of the active ingredient. The capsules are washed and dried.

Tablets: A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet, appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution: A parenteral composition suitable for administration by injection is prepared by stirring sufficient active ingredient in deionized water and mixed with, e.g., up to 10% by volume propylene glycol, salts and/or water to deliver a composition, whether in concentrated or ready-to-use form. Given the nature of the odd-chain fatty acids (alone, partially or fully-soluble in water) the amount and final concentration of the odd-chain fatty acids may be varied such that the liquid may be provided intravenously using syringes and/or standard intravenous liquids or fluids. The solution will generally be made isotonic with sodium chloride and sterilized using, e.g., ultrafiltration.

Suspension: An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

Mini-tablets: For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits: The present invention also includes pharmaceutical kits useful, for example, for providing an immediate source of alternative cellular energy, e.g., before, during or after surgery. The dosage will generally be prepared sterile and ready-to-use, e.g., one or more containers that may be broken (e.g., sealed glass ampoules), pierced with a syringe for immediate administration or even a pressurized container. Such kits may further include, if desired, one or more of various conventional pharmaceutical kit components, such as, for example, containers with one or more pharmaceutically acceptable diluents, carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Pharmaceutical Dosage Forms: The odd-chain fatty acids of the present invention may be provided in liquid form or may also be provided in a capsule, gelcap or other encapsulated form. Generally, one composition of the present invention is prepared by adding, e.g., half of the Kaolin clay or other carrier into the blended followed by addition of a first active salt form, e.g., the salt form that is less soluble in the final liquid suspension, e.g., as an emulsion in water. This process is particularly suitable for very large mixtures, e.g., 500, 1,000, 3,000 or even 5,000 liters.

One particular method of delivery of the odd-chain fatty acids of the present invention is in a tablet, capsule or gelcap that is coated for enteric delivery. Enteric coating relates to a mixture of pharmaceutically acceptable excipient(s) that is/are applied to, combined with, mixed with or otherwise added to a carrier to deliver the medicinal content, in this case one or more odd-chain fatty acids (e.g., C5, C7, C9, C11, C13 and/or C15, mixtures and combinations thereof) through the stomach unaltered for delivery into the intestines. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; easy application as a coating (substrate friendly); and economical practicality. Methods for enteric coating are well known in the art.

Remington's Pharmaceutical Sciences, discloses that enteric polymer carries generally include carboxyl groups and hydrophobic groups in the molecule and the enteric polymer is dissolved in a solvent having a specific pH value through the dissociation of the carboxyl groups. For instance, commercially available hydroxypropylmethyl cellulose acetate succinate is a derivative of hydroxypropylmethyl cellulose, which is substituted with carboxyl groups (succinoyl groups) and hydrophobic groups (acetyl groups). Alginic acid, sodium alginate other natural materials may also be used to provide an enteric coating.

Other additives and excipients may then be added to the formulation of the partially water soluble carrier-active odd-chain fatty acids mixture, e.g., adding Povidone (e.g., Povidone 30), Xantham gum (or other gums) and Sorbitol to a mixture of Kaolin Clay to provide a specific example of one formulation of the present invention. As will be apparent to those of skill in the art, the actual amount of the partially-excipient soluble active salt (e.g., non or partially water soluble) may be varied in accordance with the dissolution characteristics of the active, which may be further varied by addition of agents that affect the solubility and/or dissolution of the active in, e.g., water. As regards a pediatric formulation, the amount of active may be reduced in accordance with the dosage form approved for pediatric use.

One example of a liquid odd-chain fatty acid(s) pharmaceutical composition may be prepared for enteral or parenteral use with the following components:

| Components | Weight |
|---|---|
| Odd-chain fatty acid(s)/triglyceride | 1.0 Kg |
| emulsifier (e.g., Imwitor 375) | 100 gr |
| Purified water (USP) | 2.0 Kg |

The formulation may further include, e.g.:

| | | |
|---|---|---|
| Glycerin (USP) | 500.0 | ml |
| Sorbitol Solution, 70% (USP) | 500.0 | ml |
| Saccharin Sodium (USP) | 10.0 | gr |
| Citric Acid (USP) | 10.0 | gr |
| Sodium Benzoate (NF) | 6.0 | gr |
| Kollidon 30 | 330.0 | gr |
| Xanthan Gum 200 Mesh | 20.0 | gr |
| Bubble Gum Flavor | 11.1 | gr |
| Methylparaben | 1.0 | gr |
| Proplyparaben | 100 | mg |
| Propylene Glycol (USP) | 75 | ml |
| Additional ddH₂O | QS to 5 | liters. |

With appropriate increases of the above for scale-up.

A batch of mixed release odd-chain fatty acids in an enveloped preparation on a carrier, e.g., beads, may be prepared with the following components:

| Components | Weight |
|---|---|
| Emulsified odd-chain fatty acids/triglyceride | 8.0 mg |
| Carrier | 51.7 mg |
| Calcium Stearate | 4.0 mg |
| Talc | 4.0 mg |
| Pharmaceutical Glaze | 5.5 mg |

When combining odd-chain fatty acids (C5, C7, C9, C11, C13 and/or C15), these may be formulated as follows. A capsule for extended release of a first active and extended release of a second active in an enveloped formulation, in a single capsule:

| First Bead | Weight | | Second Bead | Weight | |
|---|---|---|---|---|---|
| odd-chain fatty | 6.0 | mg | odd-chain fatty | 2.0 | mg |
| acid C7 | | | acid C15 | | |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |
| Capsule | 1 | | | | |

When combining the odd-chain fatty acids, these may be formulated as follows. A capsule for extended release of a first active and extended release of a second active in an enveloped formulation, in a single capsule:

| First Bead | Weight | | Second Bead | Weight | |
|---|---|---|---|---|---|
| odd-chain fatty | 6.0 | mg | odd-chain fatty | 2.0 | mg |
| acid C9 | | | acids C11 | | |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |
| Mini-capsule | 1 | | | | |

A formulation for extended release of odd-chain fatty acids of a second active in an enveloped formulation, in a gelcap:

| Component | Weight | | Component | Weight | |
|---|---|---|---|---|---|
| odd-chain fatty | 6.0 | mg | odd-chain fatty | 2.0 | mg |
| acid C13 | | | acid C15 | | |
| Bead | 162.9 | mg | Bead | 108.5 | mg |
| Lacquer | 6 | mg | Lacquer | 3.3 | mg |
| Talc | 12.6 | mg | Talc | 5 | mg |
| Calcium Stearate | 12.6 | mg | Calcium Stearate | 5 | mg |
| Gelcap | 1 | | | | |

A formulation for rectal release of odd-chain fatty acids in a suppository:

| Component | Weight | |
|---|---|---|
| Odd-chain fatty acids | 100 | mg |
| Carrier | 10 | mg |
| Talc | 12.6 | mg |
| Calcium Stearate | 12.6 | mg |
| beeswax/glycerol | 1-2 | gr |

An enteric-coated soft gelatin capsule that includes the odd-chain fatty acids (with or without an emulsifier) is made by coating the odd-chain fatty acids with a lipophilic material to obtain granules, mixing the granules obtained in step with an oily matrix, antioxidants and preservatives to form a lipid suspension, mixing the lipid suspension within a soft gelatin film, and coating the soft gelatin film to obtain an enteric coated soft gelatin capsule.

The odd-chain fatty acid(s), stearic acid and triethanolamine are heated and mixed to form an emulsified fluid. The resulting emulsified fluid is mixed well by a homogenizer to obtain an emulsified suspension and enterically coated. Examples of formulations include:

| Component | Weight |
|---|---|
| Odd-chain Fatty Acids | 360.0 g |
| Stearic acid | 78.6 g |
| Ethanolamine | 21.4 g |
| Odd-chain Fatty Acids | 360.0 g |
| Stearic acid | 30.0 g |
| Triethanolamine | 20.0 g |
| Odd-chain Fatty Acids | 400.0 g |
| Stearic acid | 77.0 g |
| Ethanolamine | 23.0 g |
| Cetyl alcohol | 50.0 g |
| Odd-chain Fatty Acids | 245.0 g |
| Stearic acid | 38.5 g |
| Ethanolamine | 11.5 g |
| Cetyl alcohol | 50.0 g |
| Carboxymethyl cellulose | 25.0 g |

Adult polyglucosan disease (APBD) is a rare progressive neurogenetic disorder characterized by onset in the 4th or 5th decade of life of neurogenic bladder and progressive difficulty walking with sensory abnormalities in the lower extremities.¹⁻³ Dementia of the frontal lobe type, cerebellar abnormalities and seizures may occur in some patients.^{4,5} The motor and sensory abnormalities are caused by a myelopathy combined often with a peripheral neuropathy.⁶ After about a decade of disease progression most patient lose the ability to walk independently and in the years that follow the weakness progressively involved the trunk and the upper extremities. The disease often leads to premature death.^{3,7} No muscle or liver dysfunction has been reported to date in patients with APBD. Brain MRI typically shows extensive white matter abnormality in the cerebrum and brainstem along with atrophy of the spinal cord.^{3, 8-12}

The pathological hallmark of this disease is the accumulation of intracellular polyglucosan bodies in central (both neurons and glia) and peripheral nervous system cells but also in muscle and skin tissue.^{1, 4, 13-16} The neuron perikarya of the CNS are notably spared. These polyglucosan bodies consist of amylopectin-like polysaccharide. These findings led to the discovery that many of these patients suffer from an allelic form of glycogen storage disease type IV (GSD IV) caused by brancher enzyme (GBE1) deficiency (MIM 232500).¹⁷⁻²⁰ Contrary to children with GSD IV who generally have no residual GBE1 enzyme activity, patients with APBD and GBE1 deficiency typically have about 10% residual enzyme activity.^{18, 21} The vast majority of patients with GBE1 deficiency are of Ashkenazi Jewish (AJ) ancestry.^{3, 19, 20} Interestingly, a number of patients with reduced brancher enzyme activity and APBD have been found to be heterozygote for the most common AJ mutation (Lossos et al unpublished data).¹⁵ These patients usually have residual GBE1 activity similar to those with mutations identified on both alleles although higher activity has been reported.¹⁵ It is not known whether these are manifesting heterozygotes or whether the abnormality in the other allele was simply not found.

Existing therapy: APBD has no known effective treatment that reverses or even slows the progression of the disease.³

Mechanism of disease: The mechanism by which GBE1 deficiency causes a neurological disorder is not known. Based on the observation that polyglucosan bodies often occupy most of the diameter of axons, it was hypothesized that these inclusions mechanically disrupt normal cellular function such as intra-cellular transport^{1, 15} However, no evidence for such a mechanism has been published.

Studies described in the instant invention advance the hypothesis that at least part of the pathology in APBD is the presence of mostly abnormally branched glycogen causing dysregulation of glycogen utilization and consequent energy deficit in nervous system cells. Therefore, anaplerotic therapy comprising triheptanoin may supply nutrients to the citric acid cycle to augment cellular energy production thus preventing or reversing cellular damage in glia and neuronal cells.^{22, 23}

The hypothesis described hereinabove is based on the fact that energy deficit as manifested by hypoglycemia or exercise intolerance is a common mechanism in the glycogen storage diseases in general including in childhood GSD IV.¹⁷ Norwegian forest cats with GBE1 deficiency (and therefore a model for GSD IV) develop perinatal/neonatal hypoglycemia that causes stillbirth or death in the immediate postnatal period.^{24, 25} Since the energy requirements of newborn kittens prior to the ability to nurse depend on degradation of tissue glycogen, the presence of amylopectin-like glycogen deposits in the muscle tissue of these GBE1 deficient newborn cats suggests that in the absence of GBE1 tissue glycogen is not efficiently degraded to support energy metabolism²⁴ Affected cats may survive the critical immediate postnatal period with short-term glucose supplementation and show no obvious clinical signs until 5 months of age.²⁴ Finally, a patient with adult-onset acid maltase deficiency (Pompe disease) markedly improved on triheptanoin with a biochemical response suggesting that this C7 oil spares protein turnover in this disorder.²²

Preliminary findings in patients with APBD and GBE1 deficiency as described herein indicate that a patient who was able to performed prolonged submaximal exercise developed symptomatic hypoglycemia and an open-label study of triheptanoin supplementation in 5 patients with APBD and GBE1 deficiency showed evidence of improved motor performance as well as quality of life.

Rationale of the use of triheptanoin: Triheptanoin (glyceryl triheptanoate) is a triglyceride with odd-numbered fatty acids that is an anaplerotic substance. Anaplerotic therapy is based on the concept that there may exist an energy deficit in these diseases that might be improved by providing alternative substrates for the citric acid cycle (CAC) and therefore enhanced ATP production.^{22, 23}

After enteral absorption of triheptanoin, most of the heptanoate reaching the liver is β-oxidized to 1×anaplerotic propionyl-CoA+2×acetyl-CoA.²³ The excess acetyl-CoA and propionyl-CoA are channeled to C4- and C5-ketone bodies, which are exported from the liver to peripheral tissues.^{22, 23} The production of these ketone bodies from dietary triheptanoin occurs even when the meal contains carbohydrates. This is because the oxidation of heptanoate, a medium chain fatty acid, in liver mitochondria is not regulated by the carnitine palmitoyltransferase system, the activity of which is inhibited by dietary carbohydrates.²³ However, triheptanoin needs to provide at least 30 to 35% of total calories.²⁶ Otherwise, glucose would be the main source of energy supply and triheptanoin would not need to be oxidized. The C5-ketone bodies (3-hydroxypentanoate and 3-ketopentanoate) cross the blood brain barrier and can generate anaplerotic propionyl- and acetyl-CoA for the brain Krebs cycle.²⁷ The demonstration of the transport of C5-ketone bodies across the blood-brain barrier was provided by the treatment of a patient with pyruvate carboxylase deficiency, where cerebral anaplerosis is primarily impaired.27 The availability of C5-ketone bodies for cerebral anaplerosis was demonstrated by the normalization of glutamine and GABA in the CSF of the patient in the study, as well as the absence of brain pathology.²⁷ Anaplerotic dietary therapy with triheptanoin has been used in clinical trials to promote energy production in patients with apparent insufficiency in Krebs cycle function.^{22, 26-28} The availability of anaplerotic substrates for the brain and peripheral nervous system will allow testing the hypothesis that anaplerotic therapy can slow down or even reverse the ABPD neurodegenerative process.

APBD due to GBE deficiency is a very rare progressive degenerative neurological disorder that has no known effective treatment. The present study advances the hypothesis that decreased glycogen degradation leads to energy deficit in glia and neurons. Therefore, anaplerotic therapy, i.e. compounds providing intermediates to the citric acid cycle, may augment cellular energy production thus preventing or reversing cellular damage. The present inventors hypothesize that treatment with triheptanoin will stop or reverse the neurological progression of APBD compared to control oil that has long chain fatty acids. Therefore, the success of the therapeutic approach described herein would be the first therapy for a devastating and mostly likely underdiagnosed disease.

Use of triheptanoin in animal models: There is currently no animal model of ABPD with GBE1 deficiency. The principle of anaplerosis has been shown in isolated rat heart.²⁹ The mechanical performance of isolated rat heart decreases rapidly when the perfusate contains only precursors of acetyl-CoA, i.e., acetate or acetoacetate. Recovery of cardiac mechanical performance follows the addition of an anaplerotic substrate (pyruvate, propionylcarnitine) to the perfusate.^{30, 31} Short-term studies were conducted in rats to determine the metabolism of triheptanoin.³²

Use of triheptanoin in humans: After ingestion of triheptanoin, peripheral tissues receive two precursors of propionyl-CoA, i.e., heptanoate and C5-ketone bodies. C5-, like C4-, ketone bodies are natural substrates for the brain and can target physiological monocarboxylate transporters at the surface membrane of the blood-brain barrier.^{33, 34} Brain uptake of ketone bodies has been demonstrated in humans.³⁵⁻³⁷ Uptake of ketone bodies by diffusion or via the monocarboxylate transporters has been demonstrated in rate neurons and glia.^{38, 39}

Triheptanoin has been safely and effectively used for the treatment of long chain fatty acid oxidation defects and patients with adult-onset carnitine palmitoyltransferase II deficiency.^{26, 28} Diet treatment with triheptanoin at 30% to 35% of total daily caloric intake resulted in decreased episodes of rhabdomyolysis, improvement in pain and cardiac function.²⁶ No propionyl overload occurred. In our institution, 78 patients have been receiving chronic triheptanoin supplementation thus far—63 with mitochondrial fat oxidation defects and 14 patients with glycogen storage diseases including 5 patients with APBD and GBE1 deficiency (unpublished data).

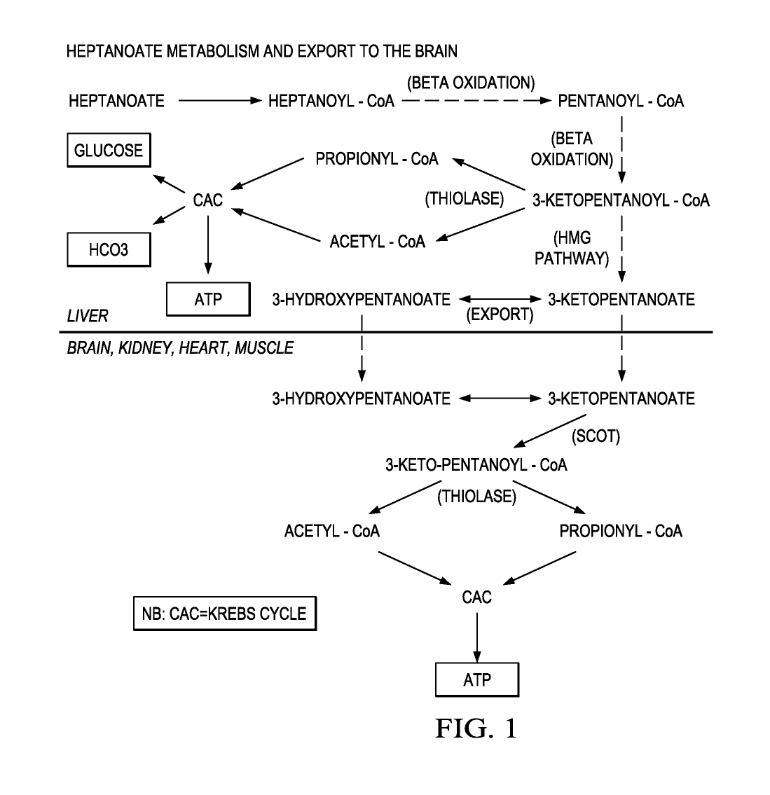

The demonstration of the transport of C5-ketone bodies across the blood-brain barrier (FIG. 1) was provided by the treatment of a patient with pyruvate carboxylase deficiency, where cerebral anaplerosis is primarily impaired.²⁷ The availability of C5-ketone bodies for cerebral anaplerosis was also demonstrated by the normalization of glutamine and GABA in the CSF of this patient, as well as the absence of brain pathology.²⁷

Use of triheptanoin in patients with APBD and GBE1 deficiency: In an open-label protocol designed by the present inventors five patients with APBD and GBE1 deficiency were been treated for a mean 8.2 months. Ages ranged from 51-66 years and all were Ashkenazi Jewish. Three patients were able to walk independently, one walked with the help of a walker and a 5^{th} patient was wheelchair bound.

The patients received triheptanoin oil (Sasol, GmbH Germany) at a dose of 1-2 g/fg/24 hours in 4 divided doses with food during 3 meals and at bedtime (representing 30-35% of total caloric intake with a control diet supplemented long chain oil (sunflower oil). The patients were randomized to either triheptanoin or control oil for 6 months. Following 6 months, the patients groups will cross over and the triheptanoin will go to control oil while the initial control oil group will receive triheptanoin both for another 6 months. The control vegetable oil (Pure Wesson soy oil) was also administered alone or as part of a meal or a snack to provide about 35% of the caloric intake

In the event that the plasma levels of propionylcarnitine increased above 8 μmol/l, the dose of triheptanoin will be reduced until the decrease of plasma propionylcarnitine is below 8 μmol/l. In the event of an organic acid abnormality such as an excessive urinary excretion of propionic and/or methylmalonic acid occur, biotin and/or vitamin B12 respectively were added to the regimen and normalization of the organic acid and acylcarnitine profile was verified. Should that not be sufficient the dose was reduced until normalization occurs. If still abnormal, patient was be excluded from the study. For GI distress, the dose will first be taken over a longer period of time (30 minutes), then fiber oligosaccharides (FOS) was used mixed with triheptanoin oil with a blender in order to facilitate GI absorption. If GI distress persisted, triheptanoin dose was reduced by 50% and re-increased progressively as the problems resolved.

Baseline evaluation based on the criteria described in Table I, herein below was performed every 3 months.

No adverse events (AE) were reported by these patients. The only AE remotely linked to triheptanoin was the rectal pain reported by one patient. Two adverse events not related to the triheptanoin were a broken ankle in one patient and wound treatment in another. There were no serious AE related to triheptanoin oil. Safety was also monitored throughout the study by urinary organic acids and blood acylcarnitine profile analyses. Changes in metabolic tests related the ingestion of triheptanoin were identified. Urinary excretion of derivatives of heptanoate oxidation were detected including pimelate, 3-hydroxypentanoate, 3-ketopentanoate, 3-hydroxypropionate, and methylcitrate—but there was no evidence of mitochondrial overload from triheptanoin-derived metabolites. In plasma, there was no substantial increase in either pentanoylcarnitine (C5) or heptanoylcarnitine (C7) but propionylcarnitine (C3) increased in most patients. These findings demonstrate that triheptanoin was catabolized completely without accumulation of secondary metabolites.

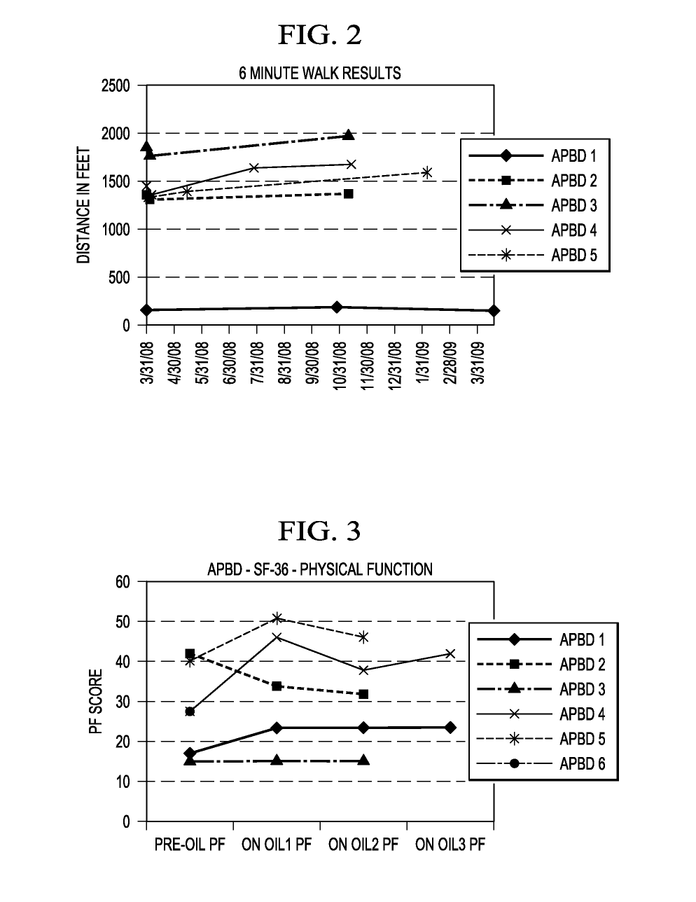

The outcome measures included: (i) the 6 minutes walk test and (ii) motion capture gait analysis and (iii) SF-36 Health Survey Questionnaire. Six minute walk test showed a mean increase of 130 feet (1246±642 to 1376±692; p=0.06). A mean improvement of 10% was observed in the 6-minute walk test over a mean follow up of 8.5 months (n=5, p=0.06). One patient had a 126 feet improvement (9.5%) at the 25 months time point. Maximal improvement seemed to occur within the first 6 months of treatment (FIG. 2). Gait analysis showed improvement over this period of time in cadence, support time, stride length, step length and walking speed of the 3 patients who were able to walk unaided. SF-36 Health Survey Questionnaire scores tended to improve in parallel with motor score (FIG. 3). Physical Function score increased in 4/5 patients on the SF-36 health survey questionnaire.

TABLE I
Baseline evaluation criteria.

| | Concomitant medication |
|---|---|
| Informed Consent | assessment |
| Vital signs | 6-minute walk test |
| Weight | Motion capture gait analysis |
| Height (baseline only) | SF-36 Health Survey |
| | Questionnaire |
| Physical and neurological | Dietary Assessment and |
| examination | education |
| Serum chemistry laboratory tests^{a} | |
| Blood acylcarnitine profile^{b} | |
| Quantitative urine organic acid | |
| analysis^{c} | |
| AE assessment | |

^{a}Serum chemistry laboratory tests (Comprehensive Metabolic Panel): Na, K, Cl, total CO₂, total Ca²⁺, creatinine, blood urea nitrogen (BUN), glucose, albumin, total protein, total bilirubin, alkaline phosphatase, alanine aminotransferase (ALT), and aspartate aminotransferase (AST). Creatine kinase
^{b}Each patient can expect a visit of up to 5 days for the initial investigation. Clinical and laboratory assessment will be carried out with whatever diet they were receiving on admission. They will then receive the diet containing triheptanoin or control oil (1-2 grams/Kg/24 hours) for the remainder of the visit with evaluation of urine organic acids and blood acylcarnitines two days after triheptanoin (or control oil) initiation which will reflect the need, if any, for supplemental biotin or cyanocobalamin

Study Design and Statistical Procedures: This study is a double blind, cross-over, phase II clinical trial assessing the effect of triheptanoin on patients with adult polyglucosan body disease (APBD). Patients will be randomized in a 1:1 ratio to the two treatment orders (placebo followed by triheptanoin and triheptanoin followed by placebo) and will remain on each treatment for 6 months with a 3 days washout period between them.

Descriptive statistics were given overall and appropriate classifications (e.g. treatment, time, etc). Continuous variables were described by their frequency of observations, mean, median, standard deviation, minimum, and maximum values. Categorical variables were described by their frequency and percentage.

The treatment effect on the primary outcome, 6-minute walk test, will be assessed using linear mixed models to account for repeated measures. If Yᵢⱼₖ is the i^{th} patient using the j^{th} treatment (trt) at the k^{th} time point then the linear mixed model will be:

Yᵢⱼₖ=β₀+β₁*trtⱼ+β₂*timeₖ+time₃*trtⱼ*timeₖ+bᵢ+eᵢⱼₖ (1)

eᵢⱼₖ˜Normal (0,σ²) (2)

bᵢ˜Normal (0,σₛ²) (3)

The hypothesis that β₁=0 will be used to test for a triheptanoin effect using an alpha of 0.05. Although no carry-over effect was anticipated, the time and treatment by time interaction effects were still assessed to verify this assumption. If the interaction was found to be significant then the treatment effect will be assessed by each time point.

Secondary outcomes were also assessed. For continuous variables with independent observations comparisons of central tendency were made using ANOVA or Kruskal-Wallis test. For dependent observations, linear mixed model analyses were used. For categorical variables with independent observations likelihood-ratio chi-square tests were used to univariately test for differences among groups. For dependent observations McNemar's or Cochran's Q (for tables larger than 2 by 2) test were used. For multivariate analyses of binary outcomes generalized linear mixed models (assuming a binomially distributed outcome and using the logit link function) were used to account for correlated observations. A 0.05 level of significance with Bonferroni correction for multiple comparisons were used. Analyses were supplemented with appropriate graphics. SAS v9.2 was be used for the analyses.

Sample size calculations were based on a cross-over study design assuming no period or carry-over effects. The detectable difference in paired means was determined for the obtainable sample size of 18 patients with a standard deviation, correlation, alpha, and power of 667.2, 0.90, 0.05, and 0.80 respectively. The standard deviation and correlation estimates were obtained from the preliminary results. Based on these values the study is adequate powered to detect a mean difference of 209 feet between the placebo and treatment group.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof' as used herein refers to all permutations and combinations of the listed items preceding the term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Publication No. 20020102737: Diagnostic Methods for Pompe Disease and Other Glycogen Storage Diseases.

United States Patent Publication No. 20080085920: Compositions Comprising Nb-Dnj, Ne-Dnj or D-Glucaro-Delta-Lactam and their Uses for the Treatment of Pain and Other Neurological Conditions.

1. Robitaille Y, Carpenter S, Karpati G, DiMauro S D. A Distinct Form of Adult Polyglucosan Body Disease With Massive Involvement of Central and Peripheral Neuronal Processes and Astrocytes: A Report of Four Cases and A Review of the Occurrence of Polyglucosan Bodies in other Conditions Such as Lafora's Disease and Normal Ageing. Brain 1980;103:315-36.

2. Klein C J, Boes C J, Chapin J E, Lynch C D, Campeau N G, Dyck P J. Adult Polyglucosan Body Disease: Case Description of an Expanding Genetic and Clinical Syndrome. Muscle Nerve 2004; 29:323-8.

3. Klein C J. Adult Polyglucosan Body Disease. In: GeneReviews. Seattle: University of Washington 2009.

4. Bigio E H, Weiner M F, Bonte F J, White C L. Familial Dementia due to Adult Polyglucosan Body Disease. Clin Neuropathol 1997; 16:227-34.

5. Boulan-Predseil P, Vital A, Brochet B, Darriet D, Henry P, Vital C. Dementia of Frontal Lobe Type Due to Adult Polyglucosan Body Disease. J Neurol 1995; 242:512-6.

6. Cafferty M S, Lovelace R E, Hays A P, Servidei S, Dimauro S, Rowland L P. Polyglucosan Body Disease. Muscle Nerve 1991; 14:102-7.

7. Sindern E, Ziemssen F, Ziemssen T, et al. Adult Polyglucosan Body Disease: A Postmortem Correlation Study. Neurology 2003; 61:263-5.

8. Negishi C, Sze G. Spinal Cord MRI in Adult Polyglucosan Body Disease. J Comput Assist Tomogr 1992; 16:824-6.

9. Rifai Z, Klitzke M, Tawil R, et al. Dementia of Adult Polyglucosan Body Disease. Evidence of Cortical and Subcortical Dysfunction. Arch Neurol 1994; 51:90-4.

10. Berkhoff M, Weis J, Schroth G, Sturzenegger M. Extensive White-Matter Changes in Case of Adult Polyglucosan Body Disease. Neuroradiology 2001; 43:234-6.

11. Savage G, Ray F, Halmagyi M, Blazely A, Harper C. Stable Neuropsychological Deficits in Adult Polyglucosan Body Disease. J Clin Neurosci 2007; 14:473-7.

12. Schiffmann R, van der Knaap M S. Invited Article: An MRI-Based Approach to the Diagnosis of White Matter Disorders. Neurology 2009; 72:750-9.

13. Gray F, Gherardi R, Marshall A, Janota I, Poirier J. Adult Polyglucosan Body Disease (APBD). J Neuropathol Exp Neurol 1988; 47:459-74.

14. Busard H L, Gabreels-Festen A A, Renier W O, et al. Adult Polyglucosan Body Disease: The Diagnostic Value of Axilla Skin Biopsy. Ann Neurol 1991; 29:448-51.

15. Ubogu E E, Hong S T, Akman H O, et al. Adult Polyglucosan Body Disease: A Case Report of A Manifesting Heterozygote. Muscle Nerve 2005; 32:675-81.

16. Schroder J M, May R, Shin Y S, Sigmund M, Nase-Huppmeier S. Juvenile Hereditary Polyglucosan Body Disease with Complete Branching Enzyme Deficiency (Type IV Glycogenosis). Acta Neuropathol 1993; 85:419-30.

17. Kishnani P S, Koeberl D, Chen Y-T. Glycogen Storage Diseases. In: Scriver C R, Beaudet A L, Sly W S, et al., eds. Metabolic and Molecular Bases of Inherited Disease. 8th ed. New York: McGraw-Hill; 2009.

18. Lossos A, Barash V, Soffer D, et al. Hereditary Branching Enzyme Dysfunction in Adult Polyglucosan Body Disease: A Possible Metabolic Cause in Two Patients Ann Neurol 1991; 30:655-62.

19. Lossos A, Meiner Z, Barash V, et al. Adult polyglucosan Body Disease In Ashkenazi Jewish Patients Carrying the Tyr329Ser Mutation in the Glycogen-Branching Enzyme Gene Ann Neurol 1998; 44:867-72.

20. Ziemssen F, Sindern E, Schroder J M, et al. Novel Missense Mutations in the Glycogen-Branching Enzyme Gene in Adult Polyglucosan Body Disease. Ann Neurol 2000; 47:536-40.

21. Bruno C, Servidei S, Shanske S, et al. Glycogen Branching Enzyme Deficiency in Adult Polyglucosan Body Disease. Ann Neurol 1993; 33:88-93.

22. Roe C R, Mochel F. Anaplerotic Diet Therapy in Inherited Metabolic Disease: Therapeutic Potential. J Inherit Metab Dis 2006; 29:332-40.

23. Brunengraber H, Roe C R. Anaplerotic Molecules: Current and Future. J Inherit Metab Dis 2006; 29:327-31.

24. Fyfe J C, Kurzhals R L, Hawkins M G, et al. A Complex Rearrangement in GBE1 Causes Both Perinatal Hypoglycemic Collapse and Late-Juvenile-Onset Neuromuscular Degeneration in Glycogen Storage Disease Type IV of Norwegian Forest Cats. Mol Genet Metab 2007; 90:383-92.

25. Fyfe J C, Giger U, Van Winkle T J, et al. Glycogen Storage Disease Type IV: Inherited Deficiency of Branching Enzyme Activity in Cats. Pediatr Res 1992; 32:719-25.

26. Roe C R, Sweetman L, Roe D S, David F, Brunengraber H. Treatment of Cardiomyopathy and Rhabdomyolysis in Long-Chain Fat Oxidation Disorders using an Anaplerotic Odd-Chain Triglyceride. J Clin Invest 2002; 110:259-69.

27. Mochel F, DeLonlay P, Touati G, et al. Pyruvate Carboxylase Deficiency: Clinical and Biochemical Response to Anaplerotic Diet Therapy. Mol Genet Metab 2005; 84:305-12.

28. Roe C R, Yang B Z, Brunengraber H, Roe D S, Wallace M, Garritson B K. Carnitine Palmitoyltransferase II Deficiency: Successful Anaplerotic Diet Therapy. Neurology 2008; 71:260-4.

29. Russell R R, 3rd, Taegtmeyer H. Changes in Citric Acid Cycle Flux and Anaplerosis Antedate the Functional Decline in Isolated Rat Hearts utilizing Acetoacetate. J Clin Invest 1991; 87:384-90.

30. Russell R R, 3rd, Taegtmeyer H. Pyruvate Carboxylation Prevents the Decline in Contractile Function of Rat Hearts Oxidizing Acetoacetate. Am J Physiol 1991; 261:H1756-62.

31. Russell R R, 3rd, Mommessin J I, Taegtmeyer H. Propionyl-L-Carnitine-Mediated Improvement in Contractile Function of Rat Hearts Oxidizing Acetoacetate. Am J Physiol 1995; 268:H441-7.

32. Kinman R P, Kasumov T, Jobbins K A, et al. Parenteral and Enteral Metabolism of Anaplerotic Triheptanoin in Normal Rats. Am J Physiol Endocrinol Metab 2006;291:E860-6.

33. Nehlig A. Brain Uptake and Metabolism of Ketone Bodies in Animal Models. Prostaglandins Leukot Essent Fatty Acids 2004; 70:265-75.

34. Morris A A. Cerebral Ketone Body Metabolism. J Inherit Metab Dis 2005; 28:109-21.

35. Hasselbalch S G, Knudsen G M, Jakobsen J, Hageman L P, Holm S, Paulson O B. Blood-Brain Barrier Permeability of Glucose and Ketone Bodies during Short-Term Starvation in Humans. Am J Physiol 1995; 268:E1161-6.

36. Pan J W, Rothman T L, Behar K L, Stein D T, Hetherington H P. Human Brain Beta-Hydroxybutyrate and Lactate Increase in Fasting-Induced Ketosis. J Cereb Blood Flow Metab 2000; 20:1502-7.

37. Pan J W, Telang F W, Lee J H, et al. Measurement of Beta-Hydroxybutyrate in Acute Hyperketonemia in Human Brain. J Neurochem 2001; 79:539-44.

38. Tildon J T, Roeder L M. Transport of 3-Hydroxy[3-14C]butyrate by Dissociated Cells from Rat Brain. Am J Physiol 1988; 255:C133-9.

39. Tildon J T, McKenna M C, Stevenson J H, Jr. Transport of 3-hydroxybutyrate by Cultured Rat Brain Astrocytes. Neurochem Res 1994; 19:1237-42.

40. ATS statement: Guidelines for the Six-Minute Walk Test. Am J Respir Crit Care Med 2002; 166:111-7.

What is claimed is:

1. A method of alleviating symptoms, improving one or more motor skills, improving a gait, treating adult polyglucosan body disorder (APBD), or any combinations thereof in a patient, comprising the steps of:
identifying the patient in need of alleviation of symptoms, improvement of one or more motor skills, improvement of gait, treatment against the APBD, or any combinations thereof; and
administering to the patient daily a dose of triheptanoin (C7TG), wherein the C7TG can optionally be mixed in with one or more food products for oral consumption by the patient.

2. The method of claim 1, wherein the improvement in one or more motor skills and gait are selected from the group consisting of increase in unaided walking time, time in cadence, support time, stride length, step length, and walking speed.

3. The method of claim 1, wherein the patient is on a regular diet, wherein the regular diet comprises one or more sources of proteins, carbohydrates, and fats.

4. The method of claim 1, wherein the C7TG comprises 30-35% of a daily caloric intake of the patient.

5. The method of claim 1, wherein the C7TG comprises 30%, 31%, 32%, 33%, 34%, and 35% of the daily caloric intake of the patient.

6. The method of claim 1, wherein the amount of C7TG administered to the patient is 1-2 g/kg/24 hrs.

7. The method of claim 1, wherein the amount of C7TG administered to the patient is 1, 1.1, 1.2, 1.3, 1.4, 1.5, 1.6, 1.7, 1.8, 1.9, and 2.0 g/kg/24 hrs.

8. The method of claim 1, wherein the dose of C7TG is administered daily for 6-8 months.

9. The method of claim 1, further comprising the steps of:
monitoring the progression of the therapy by measuring a level of one or more metabolite markers of APBD in a body fluid of the patient;
comparing the levels of the one or more metabolites with a baseline level and a control level, wherein the baseline level is the level of the metabolites in the body fluid of the patient prior to the commencement of the treatment and the control level is the level of the metabolites in the body fluid of a healthy subject not suffering from APBD; and
continuing or terminating the therapy, altering a dose, a frequency, or both of the C7TG based on the results of the comparison of the metabolite levels.

10. The method of claim 9, wherein the body fluid is selected from the group consisting of blood, plasma, and urine.

11. The method of claim 1, wherein the C7TG is used to treat one or more disorders selected from glycogen branching enzyme deficiency disorders, Andersen disease, Forbes disease, and Danon disease.

12. A composition for alleviating symptoms, improving one or more motor skills, improving gait, treating adult polyglucosan body disorder (APBD), or any combinations thereof in a patient comprising:
triheptanoin (C7TG), wherein the C7TG is used as is or is mixed in with one or more food products for oral administration for the alleviation of symptoms, improvement of one or more motor skills, improvement of gait, treatment against APBD, or any combinations thereof in the patient; and
an optional organoleptic carrier and one or more optional additives selected from the group consisting of flavoring agents, vitamins, mineral supplements, protein supplements, coloring agents, and preservatives.

13. The composition of claim 12, wherein the improvement in the one or more motor skills and gait are selected from the group consisting of increase in unaided walking time, time in cadence, support time, stride length, step length, and walking speed.

14. The composition of claim 12, wherein the composition is administered while maintaining a regular diet in the patient.

15. The composition of claim 12, wherein the C7TG comprises 30-35% of a daily caloric intake of the patient.

16. The composition of claim 12, wherein the C7TG comprises 30%, 31%, 32%, 33%, 34%, and 35% of the daily caloric intake of the patient.

17. The composition of claim 12, wherein the amount of C7TG administered to the patient is 1-2 g/kg/24 hrs.

18. The composition of claim 12, wherein the amount of C7TG administered to the patient is 1, 1.1, 1.2, 1.3, 1.4, 1.5, 1.6, 1.7, 1.8, 1.9, and 2.0 g/kg/24 hrs.

19. The composition of claim 12, wherein the dose of C7TG is administered daily for 6-8 months.

20. The composition of claim 12, wherein the composition is used to treat one or more disorders selected from defective glycogen branching enzyme deficiency disorders, Andersen disease, Forbes disease, and Danon disease.

21. A method of alleviating symptoms, improving one or more motor skills, improving gait, treating adult polyglucosan body disorder (APBD), or any combinations thereof in a patient comprising the steps of:
identifying the patient in need of alleviation of symptoms, improvement of one or more motor skills, improvement of gait, treatment against the APBD, or any combinations thereof; and
administering to the adult patient a physiologically effective amount of a formulation orally, wherein the formulation comprises one or more odd-chain triglycerides having the general formula: 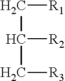
wherein, the R₁, R₂, and R₃ are esterified to the glycerol backbone are each independently fatty acids comprising odd numbered carbon chains having 5 to 15 carbon atoms, an optional organoleptic carrier, and one or more optional additives selected from the group consisting of flavoring agents, vitamins, mineral supplements, protein supplements, coloring agents, and preservatives.

22. The method of claim 21, wherein the R₁, R₂, and R₃ carbon chains are five carbons in length selected from pentanoin, triheptanoin, pentanoylcarnitine, n-pentadecanoic acid, five carbon fatty acid precursors, and derivatives thereof.

23. The method of claim 21, wherein at least one of the R₁, R₂, and R₃ carbon chains are seven carbons in length.

24. The method of claim 21, wherein the odd-chain triglyceride is triheptanoin.

25. The method of claim 21, wherein the formulation is used to treat one or more disorders selected from glycogen branching enzyme deficiency disorders, Andersen disease, Forbes disease, and Danon disease.

26. A dietary composition for providing a high fat, low carbohydrate diet to a human subject comprising:
one or more medium chain triglycerides (MCTs) having the general formula: 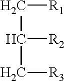
wherein, the R₁, R₂, and R₃ are esterified to the glycerol backbone are each independently fatty acids comprising odd numbered carbon chains having 5 to 15 carbon atoms;
an optional organoleptic carrier; and
one or more optional additives selected from the group consisting of flavoring agents, vitamins, mineral supplements, protein supplements, coloring agents, and preservatives.

27. The composition of claim 26, wherein the R₁, R₂, and R₃ carbon chains are five carbons in length selected from pentanoin, triheptanoin, pentanoylcarnitine, n-pentadecanoic acid, five carbon fatty acid precursors, and derivatives thereof.

28. The composition of claim 26, wherein at least one of R₁, R₂, and R₃ carbon chains are seven carbons in length.

29. The composition of claim 26, wherein the odd-chain triglyceride is triheptanoin.

30. The composition of claim 26, wherein the human subject is a healthy human subject or a human subject suffering from one or more glycogen brancher enzyme deficiency, adult polyglucosan body disorder (APBD), Andersen disease, Forbes disease, and Danon disease.

31. The composition of claim 30, wherein the composition is adapted for administration to a human subject suspected of having adult polyglucosan body disorder (APBD).

32. A dietary formulation suitable for human consumption comprising medium chain triglycerides, odd numbered carbon chain fatty acids selected from the group consisting of, five seven, and fifteen carbon fatty acids, and triglycerides thereof or both.

33. The formulation of claim 32, wherein the fatty acid is pentanoic acid.

34. The formulation of claim 32, wherein the fatty acid is heptanoic acid.

35. The formulation of claim 32, wherein the odd-chain triglyceride is triheptanoin.

36. The formulation of claim 32, wherein the composition is used to treat or alleviate the symptoms associated with one or more glycogen brancher enzyme deficiency, adult polyglucosan body disorder (APBD), Andersen disease, Forbes disease, and Danon disease.

37. The formulation of claim 36, wherein the formulation is adapted for administration to a patient with APBD.

38. The formulation of claim 32, wherein the formulation is adapted for oral administration.

39. The formulation of claim 32, wherein the formulation is adapted for enteral or parenteral administration.

40. A method of treating or alleviating symptoms in an adult patient suffering from adult polyglucosan body disorder (APBD) comprising the steps of:
identifying the adult patient in need of treatment or alleviation symptoms against APBD; and
administering a formulation of an odd-chain fatty acid comprising at least one of a C5, C7, C9, C11, C13, C15, or triglycerides thereof to the patient in a quantity sufficient to treat or alleviate the symptoms of the APBD.

41. The method of claim 40, wherein the formulation comprises one or more optional additives selected from the group consisting of flavoring agents, vitamins, mineral supplements, protein supplements, coloring agents, and preservatives.

42. The method of claim 40, wherein the formulation is adapted for parenteral, enteral, intravenous, or intramuscular administration.
